(19) United States
(12) Patent Application Publication
(10) Pub. No.: US 2025/0250229 A1
(43) Pub. Date: Aug. 7, 2025

(54) ARYLTHIO DERIVATIVES WITH PHARMACOLOGICAL ACTIVITY

(71) Applicant: IRIDEA S.R.L., Cercola (NA) (IT)

(72) Inventors: Vincenzo PAVONE, Napoli (NA) (IT); Daniele D'Alonzo, Napoli (NA) (IT); Maria De Fenza, Quarto (NA) (IT)

(73) Assignee: IRIDEA S.R.L., Cercola (NA) (IT)

(21) Appl. No.: 18/856,069

(22) PCT Filed: Apr. 18, 2023
(86) PCT No.: PCT/IB2023/053941

§ 371 Date: Oct. 11, 2024

(30) Foreign Application Priority Data
Apr. 19, 2022 (IT) 102022000007754

(51) Int. Cl. C07C 317/42; A61K 31/167; A61P 29/00

(52) U.S. Cl. CPC C07C 317/42; A61K 31/167; A61P 29/00

(57) ABSTRACT

The object of the present invention is a novel class of compounds and the pharmaceutical compositions containing them as active ingredients. The compounds according to the invention are effective in the prevention or treatment of disorders directly involving the urokinase system, its receptor and its natural inhibitor (uPA, uPAR and PAI), and/or indirectly involving its co-receptors such as formyl-peptide receptors (FPRs), integrins and vascular endothelial growth factor (VEGF).

### SUMMARY OF THE INVENTION

The object of the present invention is a novel class of compounds and the pharmaceutical compositions containing them as active ingredients. The compounds according to the invention are effective in the prevention or treatment of disorders directly involving the urokinase system, its receptor and its natural inhibitor (uPA, uPAR and PAI), and/or indirectly involving its co-receptors such as formyl-peptide receptors (FPRs), integrins and vascular endothelial growth factor (VEGF). The compounds according to the invention are effective in the prevention or treatment of a wide range of disorders such as the complications of diabetes (diabetic retinopathy, nephropathy and neuropathy), neovascular glaucoma, rubeosis iridis, age-related macular degeneration, retinopathy of prematurity, retinitis pigmentosa, irritable bowel syndrome, Crohn's disease, ulcerative rectal colitis, hereditary angioedema, hereditary haemorrhagic telangiectasia, lysosomal storage disease, rheumatoid arthritis, psoriasis, Alzheimer's disease, Parkinson's disease, coronary artery disease, destructive bone disease, endometriosis, pulmonary fibrosis, systemic sclerosis, lupus erythematosus, cancer, acute lung damage, acute respiratory distress syndrome, acute kidney injury and end-stage kidney disease.

### PRIOR ART

Invasion of the stroma by proliferating endothelial cells involves activation of proteolytic enzymes required to degrade the basement membrane of the endothelium and the extracellular matrix, thus allowing the endothelial cells to migrate through the proteins of the lysed matrix (Duran, C L. et al., 2017. Compr. Physiol. 8, 153-235). Among the protease systems involved in angiogenesis, a central role is played by a system formed by uPA, its receptor uPAR and its inhibitor PAI. Said system, in addition to regulating extracellular proteolysis by means of degradation of the extracellular matrix, also activates many intracellular signalling pathways, thus playing a broader, multi-stage role which characterises various pathological conditions, and in particular is a key factor for the invasive capacity of malignant tumours. Some of the main disorders associated with the uPA/uPAR/PA system, which also represents an effective marker for progression of said disorders, are listed below [Madunić, J., 2018. Thromb. Haemost. 118, 2020-2036; Dinesh, P. et al., 2018. Pharmacol. Res. 134, 31-39; Napolitano, F. et al., 2018. Front. Immunol. 9, 574; Rosetti, F. et al., 2016. Immunol. Rev. 269, 175-193; Rubina, K. A. et al., 2017. Arch. Dermatol. Res. 309, 433-442; Davis, J. et al., 2003. J. Biol. Chem. 278, 19054-19061; Walker, D. G. et al., 2002. Brain Res. 926, 69-79; Reuland, C. J. et al., 2020. Med. Hypotheses 138, 109602; Pan, H., et al., 2018. Exp. Ther. Med. 15, 5517-5522; Farris, S. D. et al., 2011. J. Biol. Chem. 286, 22665-22677; Schuliga, M. et al., 2018. Int. J. Biochem. Cell. Biol. 97, 108-117; Huang, J. M. et al., 2018. Front. Pharmacol. 9, 1016; Huang, W. et al., 2017. Oncotarget 8, 66951-66959; Genua, M. et al., 2016, Inflamm. Bowel Dis. 22, 2390-2401; Locri, F. et al., 2019. J. Mol. Med. 1-11; Cammalleri, M. et al., 2019. J. Cell. Mol. Med. 1-17; Marsili, S. et al., 2017. Invest. Ophthalmol. Vis. Sci. 58, 5802-5802; Rezzola, S. et al., 2017. Diabetologia 60, 719-728; Cammalleri, M. et al., 2017. Invest. Ophthalmol. Vis. Sci. 58, 3138-3148; Cammalleri, M. et al., 2016. Invest. Ophthalmol. Vis. Sci. 57, 2600-2611; Dal Monte, M. et al., 2015. Invest. Ophthalmol. Vis. Sci. 56, 2392-2407; Paueksakon, P. et al., 2002. Kidney Int. 61, 2142-2148; Hafer-Macko, C. E. et al., 2007. Neurology 69, 268-274; Kovacs, K. et al., 2015. Invest. Ophthalmol. Vis. Sci. 56, 6523-6530; Castellano, G. et al., 2018. J. Allergy Clin. Immunol. 142, 883-891; Sadick, H. et al., 2005. Haematologica 90, 818-828; DeGraba, T. et al., 2001. Ann. Neurol. 47, 229-233; Shetty, S. et al., 2007. Am. J. Physiol. Lung Cell. Mol. Physiol. 295, L967-7; Chalkias, A. et al., 2020. Mol. Diagn. Ther. 24, 517-521; Hall, A. et al., 2018. BMC Nephrology 19, 191; Zhao, Y. et al., 2015. PLoS ONE 10, e0132869; Dal Monte, M. et al., 2018. J. Cell. Mol. Med. 23, 1034-1049].

The above list demonstrates that the uPA/uPAR/PAI system represents an important therapeutic target in a number of disorders.

Many compounds which are known from the literature to interfere with the uPA/uPAR/PAI system have been proposed for the treatment of some major disorders, but have still not found a useful application as medicaments. Said compounds can be divided into three main classes: 1) inhibitors of the proteolytic activity of uPA; 2) uPAR inhibitors; 3) inhibitors of the interaction between uPAR and its co-receptors. For example, the compounds claimed in US 2004/266766, proposed as therapeutic agents for the treatment of urokinase-associated disorders, such as malignant tumours and metastasis formation, belong to the first class. The invention relates in particular to selective uPA inhibitors, based on aryl-guanidine. The compounds claimed in WO02069885, U.S. Pat. No. 6,277,818 and WO2010102253 belong to the second class. The latter are peptides that inhibit activation of uPA by uPAR, and have been proposed for the treatment of persistent or recurrent epithelial ovarian cancer, Fallopian tube cancer, primary peritoneal carcinoma, retinal oedema and age-related macular degeneration. The compounds claimed in WO2008017372, WO2017121766 and WO2017121764, claiming tetra- and penta-peptides that interfere with the uPA/uPAR/PAI system, belong to the third class. The most widely studied peptide is UPARANT [Cammalleri, M. et al., 2019. Cells 8, 925; Cammalleri, M. et al., 2017. J. Diabetes Res. Article ID 2904150, 18 pages; Rezzola, S. et al., 2017. Diabetologia 60, 719-728; Cammalleri, M. et al., 2017. Invest. Ophthalmol. Vis. Sci. 58, 3138-3148; Dal Monte, M. et al., 2018. J. Cell. Mol. Med. 1-16; Cammalleri, M. et al., 2019. J. Cell. Mol. Med. 23, 5176-5192; Cammalleri, M. et al., 2016. Invest. Ophthalmol. Vis. Sci. 57, 2600-2611; Locri, F. et al., 2019. J. Mol. Med. 97, 1273-1283; Dal Monte, M. et al., 2015. Invest. Ophthalmol. Vis. Sci. 56, 2392-2407; Carriero, M. V. et al., 2014. Mol. Cancer Ther. 13, 1092-1104; Motta, C. et al., 2016. Invest. Ophthalmol. Vis. Sci. 57, 5723-5735; Boccella, S. et al., 2017. Inflamm. Res. 66, 701-709; Rezzola, S. et al., 2017. Diabetologia 60, 719-728], which has been proposed for the treatment of diabetic retinopathy, retinitis pigmentosa, rubeosis iridis, age-related macular degeneration, macular oedema, retinopathy of prematurity and diabetic nephropathy; WO2017178333 claims retro-inverso peptides of UPARANT proposed for the treatment of melanoma and metastatic sarcoma. The cyclic peptide [SRSRY] is described for the treatment of osteosarcoma, chondrosarcoma (Ingangi, V. et al., 2016. Oncotarget 7, 54474-54487) and inflammatory bowel disease (Genua, M. et al., 2016. Inflamm. Bowel Dis. 22, 2390-2401). The common characteristic of all of said compounds is that they are cell migration and neoangiogenesis inhibitors. They interfere with the uPA/uPAR/PAI system, and are all peptide compounds, and therefore particularly expensive.

### DESCRIPTION OF THE INVENTION

The compounds according to the invention are compounds with small molecular dimensions which are easily synthesised and purified, can be manufactured at a very low cost, and are more powerful cell migration and neoangiogenesis inhibitors, as well as possessing significant anti-inflammatory activity. They are therefore specifically effective in the prevention or treatment of disorders in which the uPA/uPAR/PAI system and/or its co-receptors, such as FPRs receptors, integrins or VEGF, are directly or indirectly involved. Disorders associated with dysregulation of the uPA/uPAR/PAI system can easily be identified on the basis of abnormal values of the soluble form of uPAR (SuPAR) in biological fluids, as reported in the extensive available literature [Wu C Z, et al., Clin Biochem 2015; 48: 1324-1349; Zhang Q, et al. J Clin Lab Anal 2020; 34: e23097; Håkansson K E J, et al. Respir Res 2019; 20: 258; Enocsson H, et al. J Autoimmun 2020; 106: 102340; Garnos E, et al. PLoS One 2019; 14: e0220697; Gussen H, et al. J Intensive Care 2019; 7: 26; Frary C E, et al. Eur J Prev Cardiol 2019; 27: 570-578; van Oort P M, et al. ERJ Open Res 2019; 5: 00212-2018; Tsai P K, et al. Int J Environ Res Public Health 2019; 16: 1035; Gumus A, et al. Int J Chron Obstruct Pulmon Dis 2015; 10: 357-365; Guthoff M, et al. Sci Rep 2017; 7: 40627; Theilade S, et al. J Intern Med 2015; 277: 362-371; Eugen-Olsen J, et al. Eur J Clin Invest 2016; 46: 305-311; Okulu E, et al. J Clin Lab Anal 2015; 29: 347-352; Hoenigl M, et al. Clin Biochem 2013; 46: 225-229].

SuPAR is present in serum, but can also be found in cerebrospinal fluid, urine, saliva, or pleural, peritoneal or pericardial fluid. Quantitation of suPAR levels has also been proposed for evaluation of the severity of a number of disorders, including pneumococcal pneumonia, [Loonen A J M, et al. Eur J Clin Microbiol Infect Dis 2017; 36: 1541-1547] pneumonia in children [Wrotek A, et al. Respir Physiol Neurobiol 2015; 209: 120-123], and idiopathic pulmonary fibrosis [Johnson S, et al. Int J Radiat Biol 2020; 1-9]. SuPAR predicts a high risk of onset of acute respiratory distress syndrome in patients with sepsis, and is positively associated with inflammation and mortality [Chen D, et al. Exp Ther Med 2019; 18: 2984-2992].

By way of example, the compounds according to the invention can be used in the prevention or treatment of many disorders, such as the complications of diabetes (diabetic retinopathy, nephropathy and neuropathy), neovascular glaucoma, rubeosis iridis, age-related macular degeneration, retinopathy of prematurity, retinitis pigmentosa, irritable bowel syndrome, Crohn's disease, ulcerative rectal colitis, hereditary angioedema, hereditary haemorrhagic telangiectasia, lysosomal storage disease, rheumatoid arthritis, psoriasis, Alzheimer's disease, Parkinson's disease, coronary artery disease, destructive bone disease, endometriosis, pulmonary fibrosis, systemic sclerosis, lupus erythematosus, cancer, acute lung damage, acute respiratory distress syndrome, acute kidney injury and end-stage kidney disease. Their biological activity is more powerful than that of the products known to date, and takes place at lower concentrations.

### DETAILED DESCRIPTION OF THE INVENTION

The object of the present invention is compounds of general formula 1:

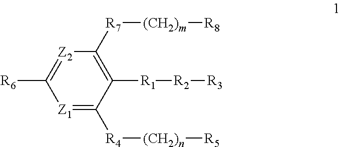

wherein:
R₁ is selected from S, O, NH, SO, SO₂ and CH(Y), wherein Y is H or an alkyl containing 1 to 9 carbon atoms;
R₂ is a CH(X) or C₂H₃X group, wherein X is H or an alkyl containing 1 to 9 carbon atoms;
R₃ is an aromatic or heteroaromatic group containing 6 to 13 carbon atoms;
R₄ and R₇, which are the same or different, are selected from NHC═O, C═ONH, C═OO, OO═C, NHC═S, C═SNH, CH═CH, CH₂ and O;
R₅ and R₈, which are the same or different, are a basic group selected from guanidine, imidazole, amino pyrimidine, amine and benzamidine;
R₆ is H or an alkyl containing 1 to 9 carbon atoms; Z, and Z₂, which are the same or different, are N, CH or C—COOH;
n and m, which are the same or different, are an integer from 1 to 5;
and the pharmaceutically acceptable salts thereof.

Examples of alkyl groups containing 1 to 9 carbon atoms are methyl, ethyl, n-propyl, isopropyl, n-butyl and tert-butyl.

Examples of aromatic groups containing 6 to 13 carbon atoms are phenyl, 1- or 2-naphthyl and indole.

Z₁ and Z₂ are preferably CH, while the basic group is preferably a guanidino group.

Compounds of general formula 1 are preferred wherein:
R₁ is S, SO or SO₂;
R₂ is CH₂, (CH₂)₂ or CH₂CH(CH₃);
R₃ is phenyl, 1-naphthyl, 2-naphthyl or indole;
R₄ and R₇, which are the same or different, are NHC═O, C═ONH, C═OO, OO═C, NHC═S, C═SNH, CH═CH, CH₂ or O;
R₅ and R₈, which are the same or different, are a basic group selected from the group consisting of guanidine, imidazole, amino pyridine, amine and benzamidine;
R₆ is H or tert-butyl;
n and m, which are the same or different, are an integer from 1 to 5;
Z₁ and Z₂, which are the same or different, are N, CH or C—COOH;

More preferred are the compounds of general formula 1 wherein:
R₁ is S, SO or SO₂;
R₂ is CH₂, (CH₂)₂ or CH₂CH(CH₃);
R₃ is phenyl, 1-naphthyl, 2-naphthyl or indole;
R₄ and R₇, which are the same or different, are NHC═O or O;
R₅ and R₈ are a guanidino group;
R₆ is H or tert-butyl;
n and in, which are the same or different, are 2 or 3;
Z₁ and Z₂, which are the same or different, are N or CH.

Even more preferred are the compounds of general formula 1 wherein:
R₁ is S, SO or SO₂;
R₂ is CH₂ or (CH₂)₂;
R₃ is phenyl, 1-naphthyl or 2-naphthyl;
R₄ and R₇, which are the same or different, are NHC═O or O;
R₅ and R₈ are guanidine;
R₆ is H or tert-butyl;
n and m, which are the same or different, are 2 or 3;
Z₁ and Z₂ are CH.

The compounds according to the invention can be synthesised by adapting known methods for that purpose, for example by adapting to the synthetic requirements the procedure described by Tew G. N. et al., 2002. Proc. Natl. Acad. Sci. U.S.A. 99, 5110-5114. They are obtained with very high yields and high purity.

Scheme 1 describes a generalised synthetic procedure designed to obtain the compounds according to the invention. In said scheme, groups Z and R and indexes n and m have the same meanings as indicated above.

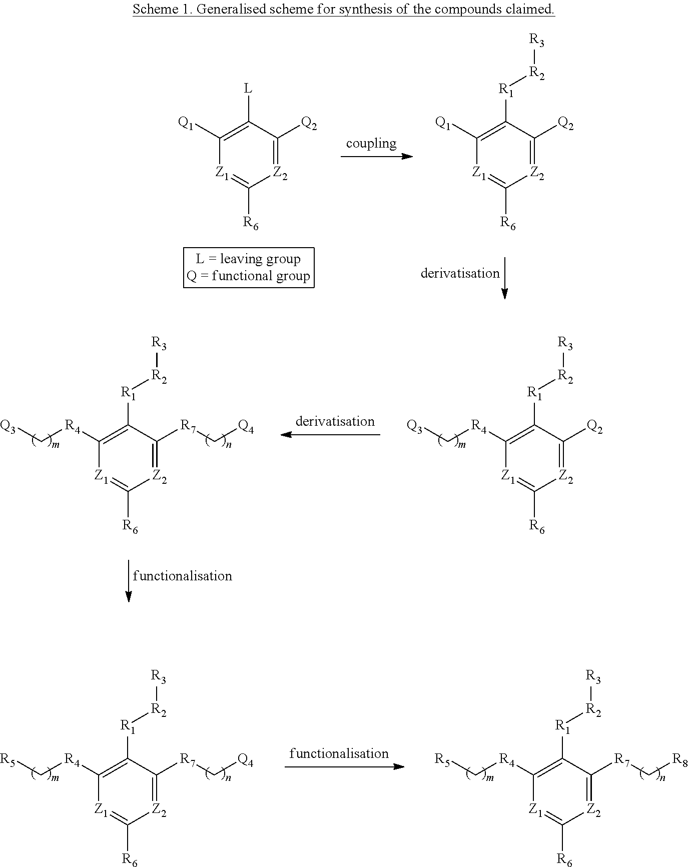

The compounds according to the invention are powerful inhibitors of cell migration and neoangiogenesis, which is exhibited at sub-fM concentrations, induced by various chemotactic stimuli such as VEGF, the peptide fMLP, the peptide SRSRY, and fetal serum, and possess significant anti-inflammatory activity.

The compounds according to the invention can therefore prevent and treat many disorders wherein the uPA/uPAR/PAI system and/or its co-receptors, such as FPRs, integrins and VEGF, are directly or indirectly involved. Said uses can easily be determined by a skilled person, for example on the basis of the SuPAR levels in biological fluids.

The compounds according to the invention can be used to prevent and treat disorders such as complications of diabetes (diabetic retinopathy, nephropathy and neuropathy), irritable bowel syndrome, Crohn's disease, ulcerative rectal colitis, cancer, acute lung damage, acute respiratory distress syndrome, acute kidney injury and end-stage kidney disease, neovascular glaucoma, rubeosis iridis, age-related macular degeneration, retinopathy of prematurity, retinitis pigmentosa, hereditary angioedema, hereditary haemorrhagic telangiectasia, lysosomal storage disease, endometriosis, pulmonary fibrosis, systemic sclerosis, lupus erythematosus, rheumatoid arthritis, psoriasis, Alzheimer's disease, Parkinson's disease, coronary artery disease and destructive bone disease.

Even more preferably, the compounds according to the invention can be used to prevent and treat complications of diabetes (diabetic retinopathy, nephropathy and neuropathy), irritable bowel syndrome, Crohn's disease, ulcerative rectal colitis, cancer, acute lung damage, acute respiratory distress syndrome, acute kidney injury and end-stage kidney disease, neovascular glaucoma, rubeosis iridis, age-related macular degeneration, retinopathy of prematurity, retinitis pigmentosa, hereditary angioedema, hereditary haemorrhagic telangiectasia and lysosomal storage disease.

The compounds according to the invention are highly effective in the prevention and treatment of disorders involving the uPA/uPAR/PAI system, in particular compared with the known compounds inhibiting interaction between uPAR and its co-receptors (WO2008017372, WO2017121766, WO2017121764, WO2017178333, WO02069885, U.S. Pat. No. 6,277,818, WO2010102253, Ingangi, V. et al., 2016. Oncotarget 7, 54474-54487; Genua, M. et al., 2016. Inflamm. Bowel Dis. 22, 2390-2401). Unlike the known compounds, the compounds according to the invention do not consist of alpha-aminoacids, and can therefore be manufactured on an industrial scale at much lower costs. Their chemical structure also obviates the well-known drawbacks of the currently known peptide compounds, such as low plasma half-life, breakdown by protease, heat and pH instability, and immunogenicity.

The compounds according to the invention are also structurally very different from uPA proteolytic activity inhibitors based on aryl guanidine (US 2004/266766), and from uPAR activation inhibiting compounds (WO02069885, U.S. Pat. No. 6,277,818, WO2010102253).

For the proposed therapeutic uses, the compounds according to the invention can be formulated as such, or in the form of salts, in pharmaceutical compositions for oral, parenteral, topical, aerosol or transdermal administration, optionally combined with other active ingredients. The unit doses in humans can vary within a wide range, typically from 0.1 μg to 1 g per dose, preferably between 0.1 mg and 100 mg, which can easily be determined by the skilled person according to the disorder to be treated, its severity and the patient's condition, in particular weight, sex and age.

The following examples illustrate the invention in greater detail.

ABBREVIATIONS: uPA—Urokinase-type Plasminogen Activator; uPAR—Urokinase-type Plasminogen Activator Receptor; PAI—Plasminogen Activator Inhibitor; FPRs—Formyl Peptide Receptors; VEGF—Vascular endothelial growth factor; suPAR—Soluble Urokinase-type Plasminogen Activator Receptor; ESI-MS—Electrospray lonisation Mass Spectrometry; NMR—Nuclear Magnetic Resonance Spectroscopy; TFA—Trifluoroacetic acid; DMSO—Dimethyl sulfoxide; ACN—Acetonitrile; HPLC—High-Performance Liquid Chromatography; FCS/FBS—Fetal bovine/calf serum; DMEM—Dulbecco's Modified Eagle's Medium; EBM—Basal Medium Eagle; HUVEC—Human umbilical vein endothelial cells; CAM—Chorioallantoic membrane; PBS—Phosphate-buffered saline.

### Example 1

Preparation of 2,6-N,N′-di(3-guanidyl)-propanamide-4-t-butyl-1-(2-phenylethyl)-sulphanyl-benzene (IRI-N-19001), the compound of general formula 1, wherein R₁ is S; R₂ is (CH₂)₂; R₃ is phenyl; R₄ and R₇ are NHC═O; R₅ and R₈ are guanidine; R₆ is tert-butyl; n=2; Z₁ and Z₂ are CH;
1) Preparation of 2,6-dinitro-4-t-butyl-phenyl(4-methyl)-benzenesulphonate (C₁₇H₁₈N₂O₇S; molecular weight: 394.43).

2,6-dinitro-4-t-butyl-phenol (30 g, 125 mmol) and tosyl chloride (24 g, 125 mmol) were dissolved in 450 ml of CH₂Cl₂, and di-isopropyl-ethylamine (24 mL, 138 mmol) was added to the solution. The mixture was left under stirring at room temperature for 2 hours. The solution was washed with a 10% citric acid solution followed by a saturated solution of NaCl. The organic phase was dried on Na₂S04. The solvent was removed by rotary evaporation, and the product was obtained as a slightly yellow crystalline solid with quantitative yields (46.2 g). ¹H NMR (600 MHz, CDCl₃), S: 1.40 (s, 9H), 2.50 (s, 3H), 7.39 (d, J=8.1 Hz, 2H), 7.80 (d, J=8.4 Hz, 2H), 8.11 (s, 2H). ESI-MS: calculated 395.4 ([M-H]⁺); found 432.2 ([M-K]⁺).
2) Preparation of 2,6-dinitro-4-t-butyl-1-(2-phenylethyl)sulphanyl-benzene (C₁₈H₂₀N₂O₄S; molecular weight: 360.43).

2,6-Dinitro-4-t-butyl-phenyl-(4-methyl)-benzenesulphonate (30.0 g, 76 mmol), 2-phenylethyl sulphane (10 g, 72 mmol) and di-isopropyl-ethylamine (13.3 mL, 76 mmol) were dissolved in 480 mL of CH₂Cl₂. The solution was left under stirring in a nitrogen atmosphere for 18 hours. The solution was extracted with dichloromethane and washed sequentially with 0.5M NaOH, 10% citric acid, a saturated solution of Na₂CO₃ and a saturated solution of NaCl. The organic phase was dried on Na₂SO₄. The crude product was chromatographed on silica gel (150 g; eluents: hexane and ethyl acetate, from 100:0 to 96:4), obtaining 25.1 g of a white solid (yield: 92%). ¹H NMR (600 MHz, CDCl₃), S: 1.37 (s, 9H), 2.87 (t, J=8.2 Hz, 2H), 3.17 (t, J=7.7 Hz, 2H), 7.15 (d, J=7.3 Hz, 2H), 7.19 (t, J=7.5 Hz, 1H), 7.27 (t, J=7.4 Hz, 2H), 7.79 (s, 2H). ESI-MS: calculated 360.4 ([M-H]⁺); found 383.2 ([M-Na]⁺), 399.2 ([M-K]⁺).

3) Preparation of 2,6-diamino-4-t-butyl-1-(2-phenethyl)sulphanyl benzene (C₁₈H₂₀N₂S; molecular weight: 300.46).

2,6-Dinitro-4-t-butyl-1-(2-phenethyl)sulphanyl benzene (10.0 g, 28 mmol), ammonium chloride (7.9 g, 148 mmol) and powdered zinc (14 g, 214 mmol) were suspended in 360 mL of methanol. The mixture was left under stirring in the dark, in an argon atmosphere, for 18 hours. The reaction mixture was dried by rotary evaporation, resuspended in toluene and filtered through celite (eluents: toluene and ethyl acetate, 1/1 v/v). The filtrate was dried by rotary evaporation, obtaining a white solid with a quantitative yield (8.4 g). ¹H NMR (600 MHz, DMSO-d₆), δ: 1.17 (s, 9H), 2.78 (s, 4H), 5.04 (bs, 4H), 6.04 (s, 2H), 7.16-7.21 (m, 3H), 7.24-7.30 (m, 2H). ESI-MS: calculated 301.2 ([M-H]⁺); found: 301.3.

4) Preparation of 2,6-N,N′-di(3-guanidyl)-propanamide-4-t-butyl-1-(2-phenethyl)sulphanyl-benzene (C₂₆H₃₈N₈O₂S; molecular weight: 526.70).

Boc-3-amino-propanoic acid (7.6 g, 40 mmol) was dissolved in anhydrous tetrahydrofuran (475 mL) and cooled to −78° C. with a dry ice/acetone bath, and isobutyl chloroformate (6.2 mL, 48 mmol) was added slowly to the solution. The mixture was left under stirring for one hour. 2,6-diamino-4-t-butyl-1-(2-phenylethyl)sulphanyl benzene (6.0 g, 20 mmol) and triethylamine (17.6 mL, 120 mmol) were added at the same temperature, and the reaction was left under stirring for 18 hours, gradually increasing the temperature to room temperature. The reaction mixture was dried with a rotary evaporator, and the residue was resuspended in 95% trifluoroacetic acid at the temperature of 0° C. to remove the Boc group. The reaction was gradually heated to room temperature, left under stirring for 2 hours, and then dried by rotary evaporation. The residue was dissolved in a mixture of water and acetonitrile (350 mL, 1/1 v/v), and 1H-pyrazole-1-carboxamidine (16.8 g, 152 mmol) was added to the suspension. The resulting mixture was left under magnetic stirring for 18 hours. The solvent was removed under low pressure. The residue was purified by reverse-phase chromatography (RP-C18, eluents: water 0.1% TFA (v/v), acetonitrile 0.1% TFA (v/v)), obtaining 5.3 g of a white solid. Yield (trifluoroacetate salt): 34%. ¹H NMR (600 MHz, H₂O/D₂O=90/10), δ: 1.24 (s, 9H), 2.61-2.65 (m, 6H), 2.86 (t, J=7.3 Hz, 2H), 3.47 (q, J=6.4 Hz, 4H), 7.09 (d, J=8.0 Hz, 2H), 7.18 (bt, J=5.8 Hz, 2H), 7.20-7.21 (m, 1H), 7.26 (t, J=7.6 Hz, 2H), 7.49 (bs, 2H), 9.53 (bs, 2H). ESI-MS: calculated 527.30 ([M-H]), found 527.15.

### Example 2

Preparation of N,N′-(5-(tert-butyl)-2-(phenethylsulphinyl)-1,3-phenylene) bis(3(guanidyl)propanamide) (IRI-N-19001-SO) (C₂₆H₃₈N₈O₃S; molecular weight: 542.7), compound of general formula 1 wherein R₁ is S═O; R₂ is (CH₂)₂; R₃ is phenyl; R₄ and R₇ are NHC═O; R₅ and R₈ are guanidine; R₆ is tert-butyl; n=2; m=2; Z₁ and Z₂ are CH;

The compound obtained in Example 1 was dissolved in the minimum amount of 30% hydrogen peroxide and maintained under stirring for 1 h. The product was freeze-dried, and the desired product was obtained quantitatively.

### Example 3

Preparation of 2,6-N,N′-di(3-guanidyl)-propanamide-4-t-butyl-1-(1-naphthyl-methyl)sulphanyl-benzene (IRI-N-19006), compound of general formula 1, wherein R₁ is S; R₂ is CH₂; R₃ is 1-naphthyl; R₄ and R₇ are NHC═O; R₅ and R₈ are guanidine; R₆ is tert-butyl; n=2; m=2; Z₁ and Z₂ are CH;

1) Preparation of naphthalen-1-yl methanethiol (C₁₁H₁₀S; molecular weight: 174.26).

Thiourea (2.28 g, 0.030 mol) was added to a solution of 1-(chloromethyl)-naphthalene (4.77 mL, 0.030 mol) in ethanol (15 mL). The mixture was heated to reflux temperature and left under magnetic stirring at the same temperature for 2 hours. The suspension was cooled to room temperature and filtered, the solid was washed several times with ethanol, and the filtrate was concentrated in the rotary evaporator until a white suspension was obtained. A solution of 4M hydrochloric acid was added until pH 2 was reached. The mixture was extracted with dichloromethane and washed with a saturated solution of NaCl. The combined organic phases were dried on Na₂SO₄ and the solvent was removed by rotary evaporation, obtaining a colourless oil with quantitative yields (5.20 g). ¹H NMR (600 MHz, CDCl₃), δ: 1.84 (t, 1H), 4.12 (d, 2H), 7.3-8.1 (m, 7H).

2) Preparation of (4-(t-butyl)-2,6-dinitrophenyl)(naphthalen-1-yl-methyl)-sulphane (C₂₁H₂₀N₂O₄S; molecular weight: 396.46).

2,6-Dinitro-4-t-butyl-phenyl-(4-methyl)-benzenesulphonate (1.0 g, 2.5 mmol), naphthalen-1-yl methanethiol (442 mg, 2.5 mmol) and di-isopropyl-ethylamine (426 μL, 2.5 mmol) were dissolved in 15 mL of CH₂Cl₂. The solution was left under stirring in a nitrogen atmosphere for 18 hours. The solution was then extracted with dichloromethane and washed in sequence with 0.5M NaOH, 10% citric acid, a saturated solution of Na₂CO₃, and finally with a saturated solution of NaCl. The combined organic phases were dried on Na₂SO₄, and the solvent was removed by rotary evaporation. The crude product was chromatographed on silica gel (30 g; eluents: hexane and ethyl acetate, 100:0 to 97:3), obtaining 876 mg of a slightly yellowish solid. Yield: 88%. ¹H NMR (600 MHz, CDCl₃), δ:1.38 (s, 9H), 4.68 (s, 2H), 7.36 (t, J=8.0 Hz, 1H), 7.42 (d, J=7.0 Hz, 1H), 7.50 (t, J=7.0 Hz, 11H), 7.57 (t, J=6.9 Hz, 1H), 7.79 (s, 2H), 7.80 (d, J=8.3, 1H), 7.85 (d, J=8.1 Hz, 1H), 8.12 (d, J=8.4 Hz, 1H). ESI-MS: calculated 397.11 ([M-H]⁺), found 434.80 ([M-K]).

3) Preparation of 5-(t-butyl)-2-((naphthalen-1-yl-methyl)-thio)-benzene-1,3-diamine (C₂₁H₂₄N₂S; molecular weight: 336.50).

(4-(t-Butyl)-2,6-dinitrophenyl)(naphthalen-1-yl-methyl)-sulphane (450 mg, 1.14 mmol), ammonium chloride (323 mg, 6.04 mmol) and powdered zinc (565 mg, 8.64 mmol) were suspended in 13 mL of methanol. The mixture was left under stirring in the dark, in an argon atmosphere, for 18 hours. The reaction mixture was dried by rotary evaporation, resuspended in toluene and filtered through celite (eluents: toluene and ethyl acetate, 1/1 v/v), obtaining a slightly yellowish filtrate. The filtrate was dried by rotary evaporation, obtaining a slightly yellowish solid with a quantitative yield (406 mg). ¹H NMR (600 MHz, DMSO-d₆), δ: 1.17 (s, 9H), 4.20 (s, 2H), 5.01 (bs, 4H), 6.03 (s, 211), 7.36-7.39 (m, 2H), 7.51-7.58 (m, 2H), 7.82 (d, J=7.5 Hz, 1H), 7.92 (d, J=7.8 Hz, 1H), 8.24 (d, J=8.2 Hz, 1H). ESI-MS: calculated 337.17 ([M-H]+); found: 337.20.

4) Preparation of N,N′-(5-(t-butyl)-2-((naphthalen-1-yl-methyl)-thio)-1,3-phenylene)-bis(3-((diaminomethylene)-amino)-propanamide) (C₂₉H₃₈N₈O₂S; molecular weight: 562.74).

Boc-3-amino-propanoic acid (98 mg, 298 mol) was dissolved in anhydrous tetrahydrofuran (5 mL) and cooled to −78° C. with a dry ice/acetone bath, and isobutyl chloroformate (49 mg, 358 μmol) was added slowly to the solution. The mixture was left under stirring for one hour. 5-(t-butyl)-2-((naphthalen-1-yl-methyl)-thio)-benzene-1,3-diamine (50 mg, 149 μmol) and triethylamine (12 μL, 894 μmol) were added, and the reaction was left under stirring for 18 hours. The reaction mixture was dried with a rotary evaporator, and the residue was resuspended in 95% trifluoroacetic acid at the temperature of 0° C. The reaction was left under stirring for 2 hours, and then dried by rotary evaporation. The residue was dissolved in a mixture of water and acetonitrile (10 mL, 1/1 v/v), and 1H-pyrazole-1-carboxamidine (219 mg, 1.5 mmol) was added to the suspension. The resulting mixture was left under magnetic stirring for 18 hours. The solvent was removed under low pressure. The residue was purified by reverse-phase chromatography (RP-C18, eluents: water 0.1% TFA (v/v), acetonitrile 0.1% TFA (v/v)), obtaining 51 mg of a white solid. Yield (trifluoroacetate salt): 44%. ¹H NMR (600 MHz, H₂O/D₂O=90/10), δ: 1.20 (s, 9H), 2.16 (t, J=6.6 Hz, 4H), 3.30 (t, J=6.7 Hz, 4H), 4.17 (s, 2H), 6.62 (d, J=7.5 Hz, 1H), 7.11 (t, J=7.7 Hz, 1H), 7.48 (bs, 2H), 7.52 (t, J=8.0 Hz, 1H), 7.58 (t, J=8.0 Hz, 1H), 7.71 (d, J=8.8 Hz, 1H), 7.89 (d, J=8.7 Hz, 1H). ESI-MS: calculated 563.28 ([M-H]⁺), found 563.15.

### Example 4

The characteristics of compounds synthesized with the methods described in Examples 1 and 3 respectively are listed below.

All samples were characterized by analytical HPLC using a Vydac C8 208TP column, 5 m, 150×4.6 mm. Eluents: A (H₂O 0.1% TFA); B (ACN 0.1% TFA).

IRI-N-19001-NH2: N,N′-(5-(tert-butyl)-2-(phenethylthio)-1,3-phenylene)bis(3-aminopropanamide)

Wherein R₁ is S; R₂ is (CH₂)₂; R₃ is phenyl; R₄ and R₂ are NHC═O; R₅ and R₈ are amine; R₆ is t-butyl; n=2; m=2; Z₁ and Z₂ are CH.

Formula: C₂₄H₃₄N₄O₂S. Molecular weight: 442.6. Analytical HPLC method: 1.0-1.5 min: 10% B; 1.5-20.0 min: 10-95% B; 20.0-22.5 min: 95% B; 22.5-25.0 min: 95-10% B; 25.0-27.5 min: 10% B. Flow rate: 1.0 mL/min. HPLC retention time: 10.2 min.

Mass spectrometry analysis (ESI-MS): n/z 443.20 ([M-H]). Proton NMR spectrum: ¹H NMR (600 MHz, DMSO-d₆), δ: 1.36 (s, 9H), 2.79 (t, J=7.8 Hz, 2H), 2.84-2.86 (m, 4H), 2.98 (t, J=7.4 Hz, 2H), 3.15-3.20 (m, 4H), 7.26 (d, J=7.4 Hz, 2H), 7.30 (t, J=7.3 Hz, 1H), 7.38 (t, J=7.6 Hz, 2H), 7.82 (bs, 2H), 7.92 (bs, 4H).

IRI-N19001-NH2-SO: N,N′-(5-(tert-butyl)-2-(phenethylsulphinyl)-1,3-phenylene) bis(3-aminopropanamide), compound of general formula 1, wherein R₁ is S═O; R₂ is (CH₂)₂; R₃ is phenyl; R₄ and R₇ are NHC═O; R₅ and R₈ are amine; R₆ is t-butyl; n=2; m=2; Z₁ and Z₂ are CH;

Formula: C₂₄H₃₄N₄O₃S. Molecular weight: 458.6. Analytical HPLC method: 1.0-1.5 min: 10% B; 1.5-20.0 min: 10-95% B; 20.0-22.5 min: 95% B; 22.5-25.0 min: 95-10% B; 25.0-27.5 min: 10% B. Flow rate: 1.0 mL/min. HPLC retention time: 9.51 min. Mass spectrometry analysis (ESI-MS): m/z 459.20 ([M-H]⁺). Proton NMR spectrum: ¹H NMR (600 MHz, DMSO-d₆), δ: 1.35 (s, 9H), 2.74 (t, J=6.7 Hz, 4H), 3.08-3.20 (m, 6H), 3.47-3.52 (m, 1H), 3.75-3.79 (m, 1H), 7.34 (t, J=7.0 Hz, 1H), 7.37 (d, J=7.3 Hz, 2H), 7.43 (t, J=7.6 Hz, 2H), 7.97 (bs, 6H).

IRI-N-19001: N,N′-(5-(tert-butyl)-2-(phenethylthio)-1,3-phenylene)bis(3-((diaminomethylene)amino) propanamide), compound of general formula 1, wherein R₁ is S; R₂ is (CH₂)₂; R₃ is phenyl; R₄ and R₇ are NHC═0; R and R₈ are guanidine; R₆ is t-butyl; n=2; m=2; Z₁ and Z₂ are CH;

Formula: C₂₆H₃₈N₈O₂S. Molecular weight: 526.3. Analytical HPLC method: isocratic, 25% B (ACN 0.1% TFA); duration: 10 minutes. HPLC retention time: 5.28 min. Mass spectrometry analysis (ESI-MS): m/z 527.2 ([M-H]+). Proton NMR spectrum: ¹H NMR (600 MHz, H₂O/D₂O=90/10), δ: 1.24 (s, 9H), 2.61-2.65 (m, 6H), 2.86 (t, J=7.3 Hz, 2H), 3.47 (q, J=6.4 Hz, 4H), 7.09 (d, J=8.0 Hz, 2H), 7.18 (bt, J=5.8 Hz, 2H), 7.20-7.21 (m, 1H), 7.26 (t, J=7.6 Hz, 2H), 7.49 (bs, 2H), 9.53 (bs, 2H).

IRI-N-19001-SO: N,N′-(5-(tert-butyl)-2-(phenethylsulphinyl)-1,3-phenylene)bis(3-((diaminomethylene)amino)propanamide), compound of general formula 1, wherein R₁ is S═O; R₂ is (CH₂)₂; R₃ is phenyl; R₄ and R₇ are NHC═O; R₅ and R₈ are guanidine; R₆ is t-butyl; n=2; m=2; Z₁ and Z₂ are CH;

Formula: C₂₆H₃₈N₈O₃S. Molecular weight: 542.70. Analytical HPLC method: isocratic, 25% B (ACN 0.1% TFA); duration: 10 minutes. HPLC retention time: 3.66 min. Mass spectrometry analysis (ESI-MS): m/z 543.20 ([M-H]). Proton NMR spectrum: ¹H NMR (600 MHz, H₂O/D₂O=90/10), S: 1.29 (s, 9H), 2.63-2.66 (m, 4H), 2.97-3.02 (m, 1H), 3.06-3.10 (m, 1H), 3.34-3.39 (m, 1H), 3.43-3.50 (m, 4H), 3.64-3.69 (m, 1H), 7.27 (d, J=7.6 Hz, 2H), 7.32 (t, J=7.3 Hz, 1H), 7.38 (t, J=7.4 Hz, 2H), 7.56 (s, 2H), 10.10 (bs, 2H).

IRI-N-19025-NH2: N,N′-(5-(tert-butyl)-2-(phenethylthio)-1,3-phenylene)bis(4-(aminobutanamide), compound of general formula 1, wherein R₁ is S; R₂ is (CH₂)₂; R₃ is phenyl; R₄ and R₇ are NHC═O; R₅ and R₈ are amine; R₆ is t-butyl; n=2; m=3; Z₁ and Z₂ are CH;

Formula: C₂₆H₃₈N₄O₂S. Molecular weight: 470.7. Analytical HPLC method: 1.0-1.5 min: 10% B; 1.5-20.0 min: 10-95% B; 20.0-22.5 min: 95% B; 22.5-25.0 min: 95-10% B; 25.0-27.5 min: 10% B. Flow rate: 1.0 mL/min. HPLC retention time: 11.16 min. Mass spectrometry analysis (ESI-MS): m/z 471.25 ([M-H]⁺). Proton NMR spectrum: ¹H NMR (600 MHz, DMSO-d₆), δ: 1.35 (s, 9H), 1.94 (dt, J=14.9, 7.3 Hz, 4H), 2.54 (t, J=7.1 Hz, 4H), 2.78 (t, J=7.6 Hz, 2H), 2.96-2.99 (m, 6H), 7.25 (d, J=7.4 Hz, 2H), 7.30 (t, J=7.1 Hz, 1H), 7.38 (t, J=7.5 Hz, 2H), 7.80 (bs, 2H), 7.83 (bs, 4H).

IRI-N-19025-NH2-SO: N,N′-(5-(tert-butyl)-2-(phenethylsulphinyl)-1,3-phenylene) bis(4-aminobutanamide), compound of general formula 1, wherein R₁ is S═O; R₂ is (CH₂)₂; R₃ is phenyl; R₄ and R₇ are NHC═O; R₅ and R₈ are amine; R₆ is t-butyl; n=2; m=3; Z₁ and Z₂ are CH;

Formula: C₂₆H₃₈N₄O₃S. Molecular weight: 486.7. Analytical HPLC method: 1.0-1.5 min: 10% B; 1.5-20.0 min: 10-95% B; 20.0-22.5 min: 95% B; 22.5-25.0 min: 95-10% B; 25.0-27.5 min: 10% B. Flow rate: 1.0 mL/min. HPLC retention time: 9.93 min. Mass spectrometry analysis (ESI-MS): m/z 487.20 ([M-H]+). Proton NMR spectrum: ¹H NMR (600 MHz, DMSO-d₆), δ: 1.35 (s, 9H), 1.91 (dt, J=14.9, 7.4 Hz, 4H), 2.47 (t, J=7.1 Hz, 4H), 2.94 (t, J=7.4 Hz, 4H), 3.09-3.13 (m, 1H), 3.16-3.20 (m, 1H), 3.49-3.54 (m, 1H), 3.68-3.72 (m, 1H), 7.33-7.37 (m, 3H), 7.43 (t, J 7.6 Hz, 2H), 7.90 (bs, 6H).

IRI-N-19025: N,N′-(5-(tert-butyl)-2-(phenethylthio)-1,3-phenylene)bis(4-((diaminomethylene)amino) butanamide), compound of general formula 1, wherein R₁ is S; R₂ is (CH₂)₂; R₃ is phenyl; R₄ and R₇ are NHC═O; R₅ and R₈ are guanidine; R₆ is t-butyl; n=2; m=3; Z₁ and Z₂ are CH;

Formula: C₂₈H₄₂N₈O₂S. Molecular weight: 554.76. Analytical HPLC method: isocratic, 25% B (ACN 0.1% TFA); duration: 10 minutes. HPLC retention time: 7.24 min. Mass spectrometry analysis (ESI-MS): m/z 555.30 ([M-H]). Proton NMR spectrum: ¹H NMR (600 MHz, H₂O/D₂O=90/10), S: 1.30 (s, 9H), 1.93 (m, 4H), 2.48 (t, J=7.6 Hz, 4H), 2.71 (t, J=7.3 Hz, 2H), 2.94 (t, J=7.3 Hz, 2H), 3.24 (t, J=6.7 Hz, 4H), 7.16 (d, J=7.7 Hz, 2H), 7.21 (bs, 1H), 7.27 (t, J=7.1 Hz, 1H), 7.33 (t, J=7.5 Hz, 2H), 7.57 (s, 2H), 9.51 (bs, 2H).

IRI-N-19025-SO: N,N′-(5-(tert-butyl)-2-(phenethylsulphinyl)-1,3-phenylene)bis(4-((diaminomethylene)amino) butanamide), compound of general formula 1, wherein R₁ is S═O; R₂ is (CH₂)₂; R₃ is phenyl; R₄ and R are NHC═O; R₅ and R₈ are guanidine; R₆ is t-butyl; n=2; m=3; Z₁ and Z₂ are CH;

Formula: C₂₈H₄₂N₈O₃S. Molecular weight: 570.8. Analytical HPLC method: isocratic, 25% B (ACN 0.1% TFA); duration: 10 minutes. HPLC retention time: 4.24 min. Mass spectrometry analysis (ESI-MS): m/z 571.30 ([M-H]⁺). Proton NMR spectrum: 1H NMR (600 MHz, H₂O/D₂O=90/10), 5:1.29 (s, 9H), 1.85 (dt, J=7.4, 15.0 Hz, 4H), 2.41 (t, J=7.8 Hz, 4H), 3.01-3.06 (m, 11H), 3.11-3.16 (m, 11H), 3.19 (t, J=7.3 Hz, 4H), 3.30-3.36 (m, 1H), 3.69-3.73 (m, 1H), 7.29 (d, J=7.3 Hz, 2H), 7.32 (t, J=7.3 Hz, 11H), 7.38 (t, J=7.4 Hz, 2H), 7.56 (bs, 2H).

IRI-N-19002: N,N′-(2-(benzylthio)-5-(tert-butyl)-1,3-(phenylene)bis(3-((diaminomethylene)amino) propanamide), compound of general formula 1, wherein R₁ is S; R₂ is CH₂; R₃ is phenyl; R₄ and R₇ are NHC═O; R₅ and R₈ are guanidine; R₆ is t-butyl; n=2; m=2; Z₁ and Z₂ are CH;

Formula: C₂₅H₃₆N802S. Molecular weight: 512.68. Analytical HPLC method: isocratic, 25% B (ACN 0.1% TFA); duration: 10 minutes. HPLC retention time: 4.54 min. Mass spectrometry analysis (ESI-MS): m/z 513.10 ([M-H]*). Proton NMR spectrum: 1H NMR (600 MHz, H₂O/D₂O=90/10), δ: 1.22 (s, 9H), 2.59 (t, J=13.0 Hz, 4H), 3.46 (q, J=6.4 Hz, 4H), 3.67 (s, 2H), 6.89-6.90 (m, 2H), 7.14-7.16 (m, 3H), 7.20 (bt, 2H), 7.53 (bs, 2H), 9.25 (bs, 2H).

IRI-N-19004: N,N′-(5-(tert-butyl)-2-((naphthalen-2-ylmethyl)thio)-1,3-phenylene)bis(3-((diamino methylene)amino)propanamide), compound of general formula 1, wherein R₁ is S; R₂ is CH₂; R₃ is 2-naphthyl; R₄ and R₇ are NHC═O; R₅ and R₈ are guanidine; R₆ is t-butyl; n=2; m=2; Z₁ and Z₂ are CH;

Formula: C29H₃₈N₈O₂S. Molecular weight: 562.74. Analytical HPLC method: isocratic, 25% B (ACN 0.1% TFA); duration: 12 minutes. HPLC retention time: 10.21 min. Mass spectrometry analysis (ESI-MS): m/z 563.10 ([M-H]⁺). Proton NMR spectrum: ¹H NMR (600 MHz, H₂O/D₂O=90/10), S: 1.21 (s, 9H), 2.44 (bt, J=6.5 Hz, 4H), 3.24-3.34 (m, 4H), 3.83 (s, 2H), 7.12 (bs, 1H), 7.18 (d, J=9.3 Hz, 1H), 7.43-7.47 (m, 2H), 7.51 (bs, 2H), 7.60 (bd, J=7.3 Hz, 1H), 7.73 (d, J=8.9 Hz, 1H), 7.80 (d, J=8.8 Hz, 1H), 9.12 (bs, 2H).

IRI-N-19006: N,N′-(5-(tert-butyl)-2-((naphthalen-1-ylmethyl)thio)-1,3-phenylene)bis(3-((diamino methylene)amino)propanamide), compound of general formula 1, wherein R₁ is S; R₂ is CH₂; R₃ is 1-naphthyl; R₄ and R₇ are NHC═O; R₅ and R₈ are guanidine; R₆ is t-butyl; n=2; m=2; Z₁ and Z₂ are CH;

Formula: C₂₉H₃₈N₈O₂S; Molecular weight: 562.74; Analytical HPLC method: isocratic, 25% B (ACN 0.1% TFA); duration: 12 minutes; HPLC retention time: 8.03 min. Mass spectrometry analysis (ESI-MS): m/z 563.10 ([M-H]^{t}); Proton NMR spectrum: ¹H NMR (600 MHz, H₂O/D₂O=90/10), 5:1.20 (s, 9H), 2.16 (t, J=6.6 Hz, 4H), 3.30 (t, J=6.7 Hz, 4H), 4.17 (s, 2H), 6.62 (d, J 7.5 Hz, 1H), 7.11 (t, J 7.7 Hz, 11H), 7.48 (bs, 2H), 7.52 (t, J=8.0 Hz, 1H), 7.58 (t, J=8.0 Hz, 1H), 7.71 (d, J=8.8 Hz, 1H), 7.89 (d, J=8.7 Hz, 1H).

### Example 5

### Ex Vivo Assays of Anti-Angiogenic Activity on Rat Aorta Rings

Thoracic aortas obtained from 5- to 10-week-old male rats were cut into rings measuring about 1 mm. The rings were placed on the base of a 24-well plate (positioning the luminal axis parallel to the base of the well), and incubated with a fibrin-polymerising solution. After 5 min, VEGF (30 ng/mL) was added to the wells in an appropriate culture medium in the presence or absence of the compounds according to the invention at the concentration of 100 μM. The medium was changed three times a week as from day 3. The angiogenic response was measured by counting the number of new vessels that sprouted over time (Table 1). The endothelial filaments were counted after 6 days' incubation.

TABLE 1
Endothelial filaments (standardised for the number of
rings) obtained by incubating the rings with VEGF in
the presence or absence of the specified compounds.

| Compound | Filaments |
|---|---|
| VEGF | 17 ± 4 |
| VEGF/IRI-N-19001 | 1.0 ± 0.4 |
| VEGF/IRI-N-19002 | 1.4 ± 0.5 |
| VEGF/IRI-N-19004 | 1.2 ± 0.5 |
| VEGF/IRI-N-19025 | 1.9 ± 1.1 |

### Example 6

### In Vivo Assays on Chorioallantoic Membrane (CAM)

42 fertilised hens' eggs were disinfected with a 20% ethanol solution and incubated at a temperature of 37-38° C. and a humidity of 40-60% in a rotary egg incubator. The eggs were positioned horizontally and incubated for the next 11 days to promote embryo development.

On day 11, the position of the CAM was identified by candling for 36 eggs, the air chamber was removed, and slits made in the shell in order to position sterile paper filters saturated with the effectors at different concentrations (V=2.5 uL) on the CAM. On day 14, CAMs were photographed with a stereomicroscope (Carl Zeiss Vision GmbH). Quantitative analysis was conducted (count of vessels leading from the filter) by skeletonizing the images and using AngioTool64 0.6a software, and the percentage of neovascularisation at the various concentrations of effectors was determined (Table 2).

TABLE 2
Effect of the specified compounds on CAM neovascularisation.

| Effector | Vascularisation (%) |
|---|---|
| PBS | / |
| VEGF165-0.1 ug | 100 |
| VEGF165/IRI-N-19001-1 ug | 10 |
| VEGF165/IRI-N-19001-0.1 ug | 26 |
| VEGF165/IRI-N-19001-0.01 ug | 33 |
| VEGF165/IRI-N-19001-0.001 ug | 37 |
| VEGF165/IRI-N-19001-0.0001 ug | 45 |
| VEGF165/IRI-N-19002-1 ug | 13 |
| VEGF165/IRI-N-19002-0.1 ug | 31 |
| VEGF165/IRI-N-19002-0.01 ug | 36 |
| VEGF165/IRI-N-19002-0.001 ug | 42 |
| VEGF165/IRI-N-19002-0.0001 ug | 48 |
| VEGF165/IRI-N-19004-1 ug | 8 |
| VEGF165/IRI-N-19004-0.1 ug | 19 |
| VEGF165/IRI-N-19004-0.01 ug | 24 |
| VEGF165/IRI-N-19004-0.001 ug | 33 |
| VEGF165/IRI-N-19004-0.0001 ug | 38 |

### Example 7

In Vivo Anti-Inflammatory Activity Test Immunocompetent C57Bl/6N male mice (8-10 weeks) were infected with strains of P. aeruginosa RP73 (4-6×10⁵ CFU, Colony Forming Units), incorporated in agar microbeads. The microbeads were prepared by following the procedures described in the literature (Bayes H K, Ritchie N, Irvine S, Evans T J. A murine model of early Pseudomonas aeruginosa lung disease with transition to chronic infection. Sci Rep. 2016 Nov. 2; 6:35838. doi: 10.1038/srep35838), and they were administered to the mice by intratracheal injection. Subcutaneous administration of the compounds according to the invention at the concentration of 12 mg/kg, or of the carrier, was conducted on the mice 18 hours before inoculation of the bacterium, and repeated daily for 6 days. The general state of health of the mice was analysed once or twice a day throughout the duration of the experiment. Six days after infection, the mice were euthanised by carbon dioxide inhalation. Bronchoalveolar lavage fluid analysis (BALF) indicated a significant reduction in the number of neutrophils in the treated mice compared with those treated with the carrier only. No significant reduction in the number of macrophages was observed in the treated mice compared with those treated with the carrier only (Table 3).

TABLE 3
In vivo effect of the specified compounds (at the dose of 12 mg/kg)
on neutrophil and macrophage recruitment in C57Bl/6N mice.

| Compound | Total cells (%) | Neutrophils (%) | Macrophages (%) |
|---|---|---|---|
| IRI-N-19001 | −55 ± 12 | −61 ± 5 | −10 ± 3 |
| IRI-N-19002 | −54 ± 9 | −60 ± 6 | −9 ± 4 |
| IRI-N-19004 | −56 ± 8 | −58 ± 8 | −5 ± 4 |
| IRI-N-19025 | −50 ± 10 | −55 ± 9 | −8 ± 5 |

1. Compounds of general formula 1 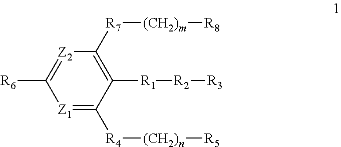
wherein:
R₁ is selected from S, O, NH, SO, SO₂ and CH(Y), wherein Y is H or an alkyl containing 1 to 9 carbon atoms;
R₂ is a CH(X) or C₂H₃X group, wherein X is H or an alkyl containing 1 to 9 carbon atoms;
R₃ is an aromatic or heteroaromatic group containing 6 to 13 carbon atoms;
R₄ and R₇, which are the same or different, are selected from NHC═O, C═ONH, C═OO, OO═C, NHC═S, C═SNH, CH═CH, CH₂ and O;
R₅ and R₈, which are the same or different, are a basic group selected from guanidine, imidazole, amino pyrimidine, amine and benzamidine;
R₆ is H or an alkyl containing 1 to 9 carbon atoms;
Z₁ and Z₂, which are the same or different, are N, CH or C—COOH;
n and m, which are the same or different, are an integer from 1 to 5;
and pharmaceutically acceptable salts thereof.

2. Compounds according to claim 1 wherein:
R₁ is S, SO or SO₂;
R₂ is CH₂, (CH₂)₂ or CH₂CH(CH₃);
R₃ is phenyl, 1-naphthyl, 2-naphthyl or indole;
R₄ and R₇, which are the same or different, are NHC═O, C═ONH, C═OO, OO═C, NHC═S, C═SNH, CH═CH, CH₂ or O;
R₅ and R₈, which are the same or different, are a basic group selected from the group consisting of guanidine, imidazole, amino pyridine, amine and benzamidine;
R₆ is H or tert-butyl;
n and m, which are the same or different, are an integer from 1 to 5;
Z₁ and Z₂, which are the same or different, are N, CH, or C—COOH.

3. Compounds according to claim 1 wherein:
R₁ is S, SO, or SO₂;
R₂ is CH₂, (CH₂)₂ or CH₂CH(CH₃);
R₃ is phenyl, 1-naphthyl, 2-naphthyl or indole;
R₄ and R₇, which are the same or different, are NHC═O or 0;
R₅ and R₈ are a guanidino group;
R₆ is H or tert-butyl;
n and m, which are the same or different, are 2 or 3;
Z₁ and Z₂, which are the same or different, are N or CH.

4. Compounds according to claim 1 wherein:
R₁ is S, SO or SO₂;
R₂ is CH₂ or (CH₂)₂;
R₃ is phenyl, 1-naphthyl or 2-naphthyl;
R₄ and R₇, which are the same or different, are NHC═O or O;
R₅ and R₈ are guanidine;
R₆ is H or tert-butyl;
n and m, which are the same or different, are 2 or 3;
Z₁ and Z₂ are CH.

5. Pharmaceutical compositions comprising a compound of claim 1 in a mixture with carriers or excipients.

6. The pharmaceutical compositions according to claim 5 for subcutaneous, intramuscular, intravenous, aerosol, intraocular, oral, nasal, sublingual, topical or transdermal administration.

7. Method of preventing or treating diseases with the compound of claim 1 in patients in need thereof, in which the uPA/uPAR/PAI system and/or its co-receptors are directly or indirectly involved, said method comprising
administering said compounds in said patients.

8. The method according to claim 7 wherein the diseases include complications of diabetes, irritable bowel syndrome, Crohn's disease, ulcerative rectal colitis, cancer, acute lung damage, acute respiratory distress syndrome, acute kidney damage and end-stage kidney disease, neovascular glaucoma, rubeosis iridis, age-related macular degeneration, retinopathy of prematurity, retinitis pigmentosa, hereditary angioedema, hereditary haemorrhagic telangiectasia, lysosomal storage disease, endometriosis, pulmonary fibrosis, systemic sclerosis, lupus erythematosus, rheumatoid arthritis, psoriasis, Alzheimer's disease, Parkinson's disease, coronary artery disease and destructive bone disease.

9. The method according to claim 7 wherein the diseases include complications of diabetes, irritable bowel syndrome, Crohn's disease, ulcerative rectal colitis, cancer, acute lung damage, acute respiratory distress syndrome, acute kidney damage and end-stage kidney disease, neovascular glaucoma, rubeosis iridis, age-related macular degeneration, retinopathy of prematurity, retinitis pigmentosa, hereditary angioedema, hereditary haemorrhagic telangiectasia and lysosomal storage disease.
